(19) United States
(12) Patent Application Publication
(10) Pub. No.: US 2013/0066230 A1
(43) Pub. Date: Mar. 14, 2013

(54) SINGLE PIECE BIOPSY FORCEPS

(71) Applicants: Changqing Li, Bloomington, IN (US); Matthew Otten, Cincinnati, OH (US)

(72) Inventors: Changqing Li, Bloomington, IN (US); Matthew Otten, Cincinnati, OH (US)

(21) Appl. No.: 13/609,875

(22) Filed: Sep. 11, 2012

Related U.S. Application Data
(60) Provisional application No. 61/534,023, filed on Sep. 13, 2011.

(51) Int. Cl. A61B 10/06; B23P 15/28; B21D 11/10

(52) U.S. Cl. 600/564; 72/362; 76/101.1

(57) ABSTRACT

Single piece biopsy forceps having jaws that are caused to open and close in response to longitudinal movement of an actuation rod. The single piece forceps are formed from a single metal blank with opposing jaw sections connected via a self-hinge. The metal blank includes a self-hinge portion, a channel section on either end of the hinge portion and a jaw section on distal ends of the channel sections. The jaws are formed from the jaw section of the metal blank and the channel sections are bent to provide generally U-shaped channel sections. The hinge portion is bent so that the jaws are oriented in opposed aligned relation. A fixed and a movable pin are disposed through slots in the channel sections. Movement of the movable pin with respect to the fixed pin via an actuator rod or wire causes the jaws of the biopsy forceps to open and close.

### CROSS REFERENCE TO RELATED APPLICATIONS

This application claims priority benefit of U.S. Provisional Application No. 61/534,023 filed Sep. 13, 2011 and titled Single Piece Biopsy Forceps.

STATEMENT REGARDING FEDERALLY SPONSORED RESEARCH OR DEVELOPMENT

Not Applicable

### BACKGROUND OF THE INVENTION

The present invention relates generally to biopsy forceps and more specifically to biopsy forceps having opposed jaw portions and an interconnecting hinge portion formed as a single piece with the jaw sections.

Biopsy forceps are typically single use devices that are disposed of following use. Given the large number of biopsy forceps used and the fact that they are typically disposed of following a single use, it is desirable for such devices to be manufacturable in high volumes at low cost.

Various designs of biopsy forceps are known in the art however most biopsy forceps designs are relatively complex multi-piece mechanical devices. It would therefore be desirable to have a design for biopsy forceps that was suitable for high volume and low cost manufacture.

### BRIEF SUMMARY OF THE INVENTION

In accordance with the present invention single piece biopsy forceps are disclosed having opposed jaw portions connected by a self-hinge. A metal blank is formed via stamping, photochemical etching or any other suitable forming operation. The metal blank has first and second jaw sections, first and second channel sections and a self-hinge section. The jaw sections are formed into cup-shaped jaws . The channel sections are bent along parallel spaced longitudinal form lines to form first and second generally U-shaped channel sections on either side of the self-hinge section. The work piece is then bent along a hinge form line that is perpendicular to the longitudinal form lines. In one embodiment, this self-hinge has a radius to provide a curved self-hinge connecting the first and second channel sections on either end of the self-hinge. In one embodiment, an opening is provided in the self-hinge section to allow for the passage of an actuation rod therethrough. The actuation rod provides linear motion along a longitudinal axis which causes the jaws to open and close as later described.

A first pair of elongated slots are provided in the sides of each channel section in opposed aligned relation to accommodate a stationary or fixed pin approximately midway between the self-hinge and the forward edge of the jaws of the forceps. The slots are angled to cause the jaws to open and close as a result of linear motion of the fixed pin within the elongated slots. A second pair of curvilinear slots are provided in the sides of each channel section in opposed aligned relation to accommodate a movable pin. The second pair of curvilinear slots are selectively positioned in the sides of the channel sections and configured to allow the jaws to close with the fixed pin extending through the first pair of slots and the movable pin extending through the second pair of slots. The actuation rod extends through the opening in the self-hinge and is coupled to the movable pin. To open and close the jaws, the actuation rod is moved linearly along a longitudinal axis perpendicular to the axis of the movable pin to produce relative motion of the movable pin with respect to the fixed pin and thereby open and close the jaws as the fixed pin slidably moves within and relative to the first pair of slots provided in the sides of the channel sections.

Other features, aspects and advantages of the above-described single piece forceps will be apparent to those of ordinary skill in the art from the Detailed Description of the Invention in conjunction with the Drawings that follow.

### BRIEF DESCRIPTION OF THE SEVERAL VIEWS OF THE DRAWINGS

The invention will be more fully understood by reference to the following Detailed Description of the Invention in conjunction with the Drawings of which:

FIG. 1 is a top plan view of a blank used to form the single piece forceps of the present invention;

FIG. 2a is a top plan view of the metal blank after forming the jaw sections of the biopsy forceps;

FIG. 2b is a top plan view of the metal blank after forming the jaw sections of the biopsy forceps and bending of first and second channel sections to form U-shaped channel sections at either end of the self-hinge-section;

FIG. 3 is a first frontal perspective view of the single piece biopsy forceps depicting the forceps with the jaws open;

FIG. 4 is a second rear perspective view of the single piece biopsy forceps depicting the forceps with the jaws open;

FIG. 5 is a third frontal perspective view of the single piece biopsy forceps depicting the forceps with the jaws closed; FIG. 6 is a fourth rear perspective view of the single piece biopsy forceps depicting the forceps with the jaws closed,

FIG. 7 is a side plan view of the single piece biopsy forceps depicting stationary and movable pins extending through respective slots of the channel sections with the jaws closed;

FIG. 8 is a side plan view of the single piece biopsy forceps depicting stationary and movable pins extending through respective slots of the channel sections with the jaws open; and

FIG. 9 is a perspective view of a single piece biopsy forceps having a sharpened blade like cutting edge on opposing jaws.

### DETAILED DESCRIPTION OF THE INVENTION

The present application incorporates by reference the entire disclosure of U.S. Provisional Application No. 61/534,023 titled Single Piece Biopsy forceps filed Sep. 13, 2011.

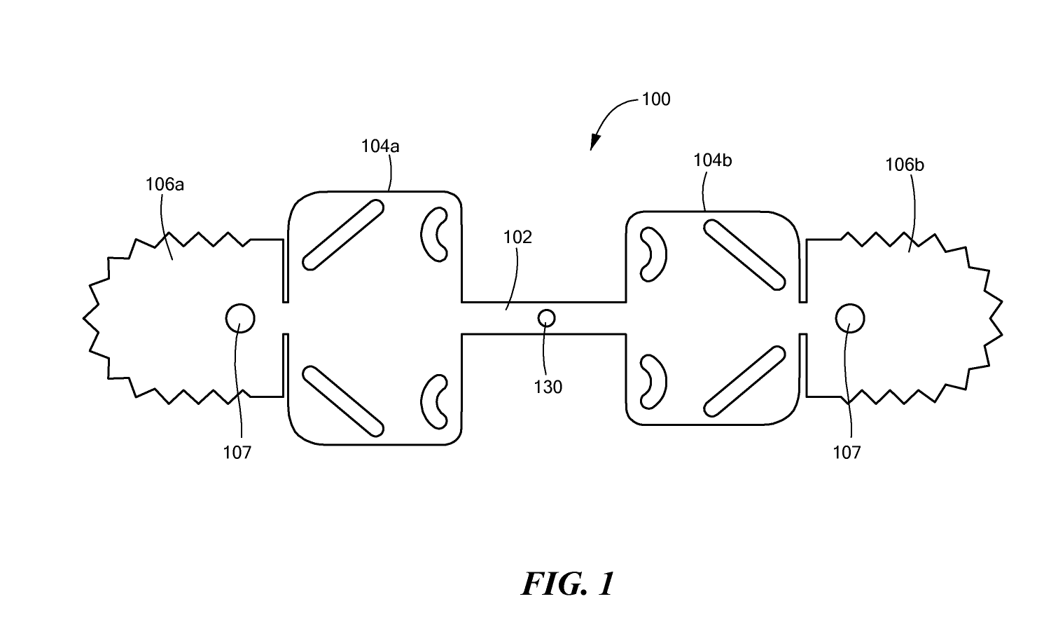

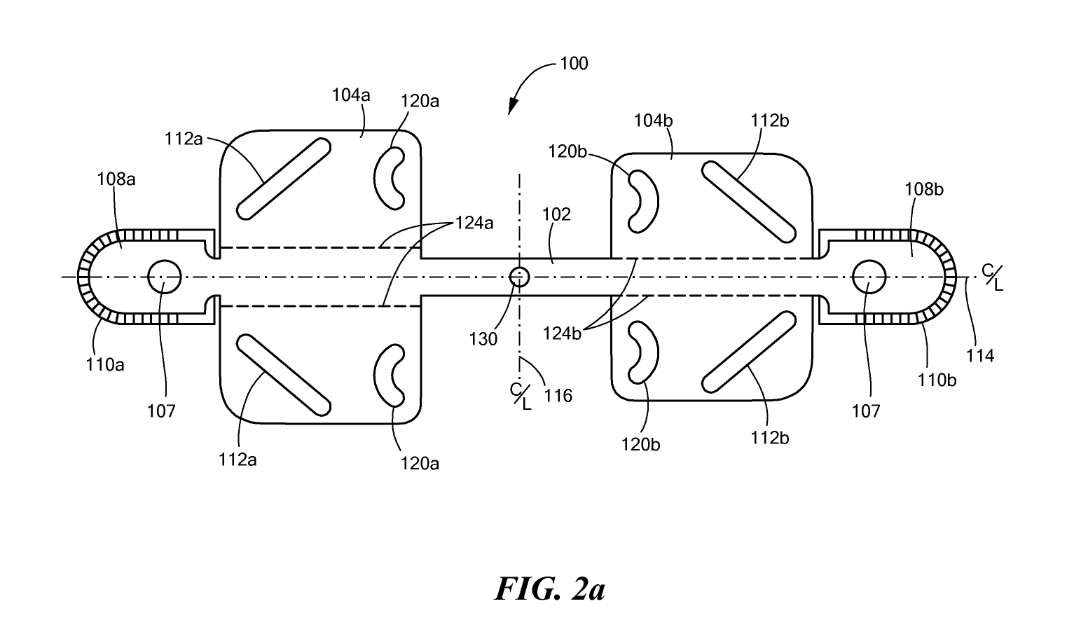

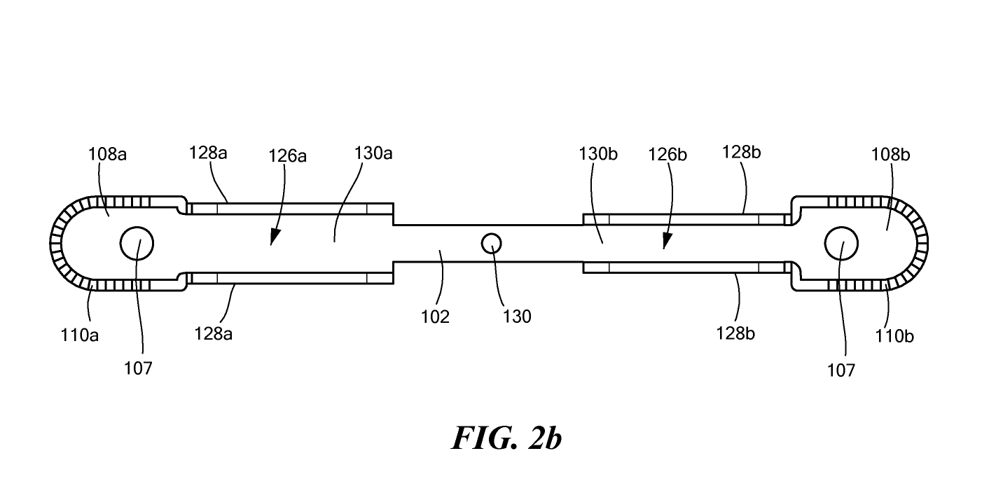

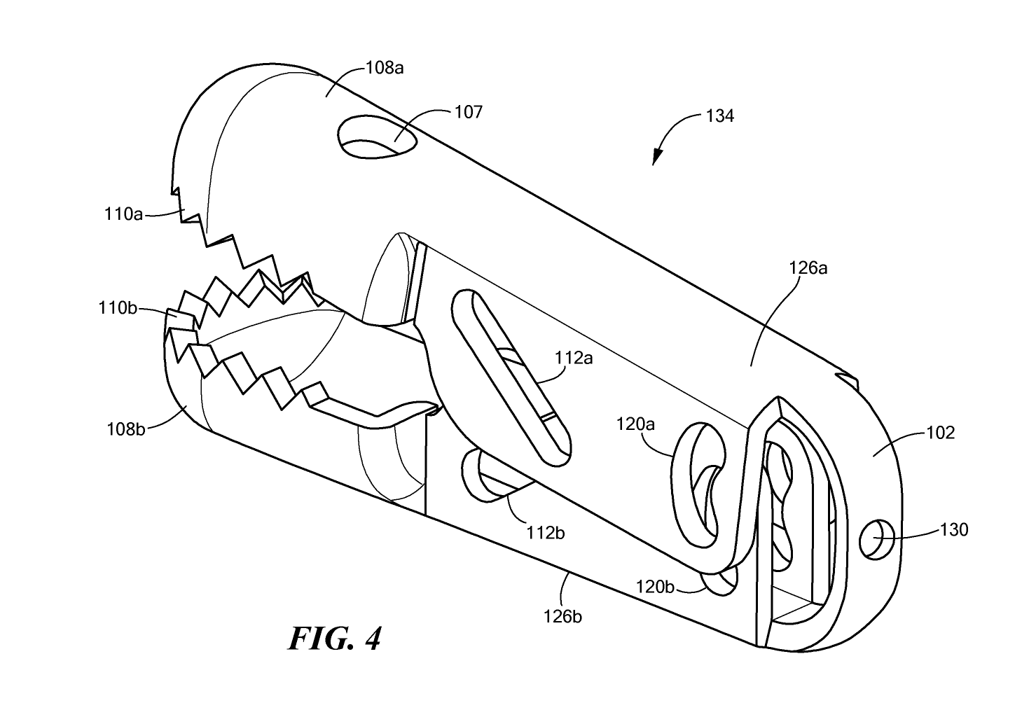

In accordance with the present invention a single piece biopsy forceps is disclosed. Referring to FIGS. 1-8, the single piece biopsy forceps is, in one embodiment, fabricated out of a single metal blank 100 such as illustrated in FIG. 1. The blank 100 has a central elongated hinge section 102, first and second channel sections 104a, 104b that extend from opposing ends of the hinge section 102 and first and second jaw sections 106a, 106b that extend from the channel sections 104a, 104b respectively. The jaw sections 106a, 106b of the metal blank 100 are formed into first and second jaws 108a, 108b (FIG. 2a). The jaws 108a, 108b have teeth 110a, 110b that are configured so as to mesh with one another when the jaws 108a, 108b are closed for cutting and retrieval of tissue. The jaws 108a, 108b may include one or more drainage openings 107.

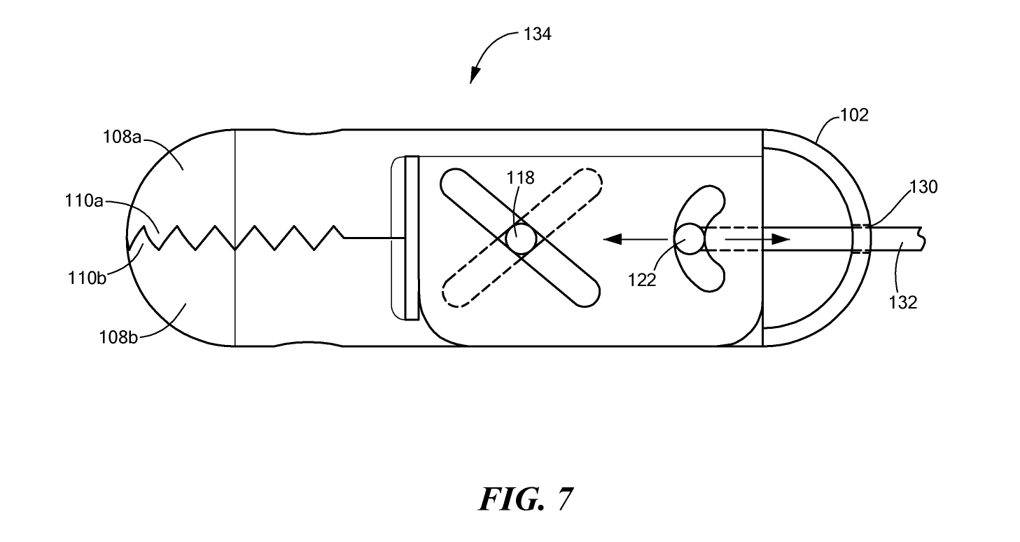

The blank 100 has first pairs of slots 112a, 112b that are disposed generally in symmetric aligned relation with respect to a first longitudinal centerline 114 extending through the metal blank 100 and additionally generally in aligned relation with respect to a second centerline 116 extending through the self-hinge section 102. that is generally perpendicular to the first longitudinal centerline 112. The first pairs of slots 112a, 112b are sized to receive a stationary or fixed pin 118 (FIG. 7)as subsequently described.

Second pairs of slots 120a, 120b are also disposed generally in symmetric aligned relation with respect to the longitudinal centerline 114 and additionally, generally in aligned relation with respect to the second centerline 116. The first pairs of slots 112a, 112b receive the fixed pin 118 following the forming operations and the second pairs of slots 118 receive a movable pin 122 following the forming operations as subsequently described. In one embodiment, the first pairs of slots 112a, 112b are elongated slots that have a central slot axis that forms an acute angle with respect to the longitudinal centerline 114. The second pairs of slots 120a, 120b are curvilinear slots as illustrated.

It should be recognized that the order of the forming operations described herein may be varied. Referring to FIGS. 1 and 2a, the jaw sections 106a, 106b of the metal blank 100 are formed into jaws 108a, 108b via any suitable metal forming technique known in the art.

As shown in FIG. 2a, form lines 124a in channel section 104a are spaced symmetrically about the longitudinal centerline 114 by a first distance and form lines 124b in channel section 104b are spaced symmetrical about the longitudinal centerline 114 by a second distance that is slightly less than the first distance.

The channel section 104a is bent along longitudinal form lines 124a in a metal forming operation to form a generally U-shaped channel section 126a having opposed side portions 128a. The channel section 104b is bent along longitudinal form lines 124b to form a generally U-shaped channel section 126b having opposed side portions 128b. The side portions 128a, 128b are generally orthogonal to the central portions 130a, 130b of the respective U-shaped channel sections.

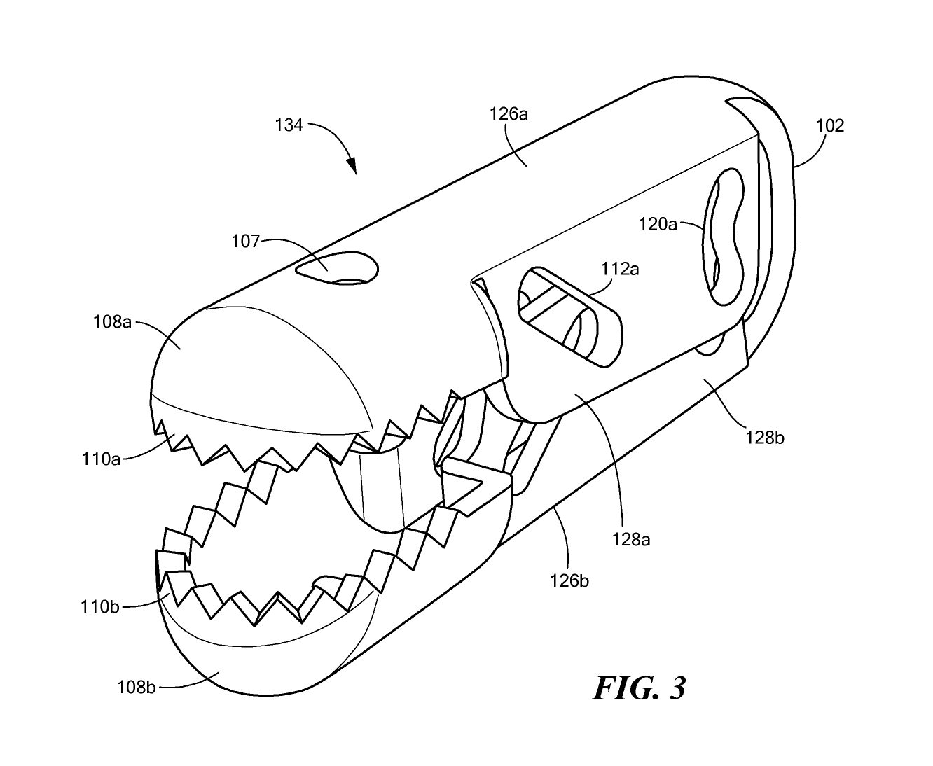

Following the formation of the generally U-shaped channel sections 126a, 126b (FIG. 2b), the pairs of slots 112a. 120a, 112b, 120b are disposed in the side portions 128a, 128b of respective U-shaped channel sections 126a, 126b with each pair of slots in opposed aligned relation. The dimension between the inner surfaces of the side portions 128a is slightly greater than the distance between the outer surfaces of the side portions 128b to permit the channel section 126b to move slidably within side portions 128a once the metal blank is bent around the self-hinge centerline 116 for form the biopsy forceps 134 (FIGS. 3-6).

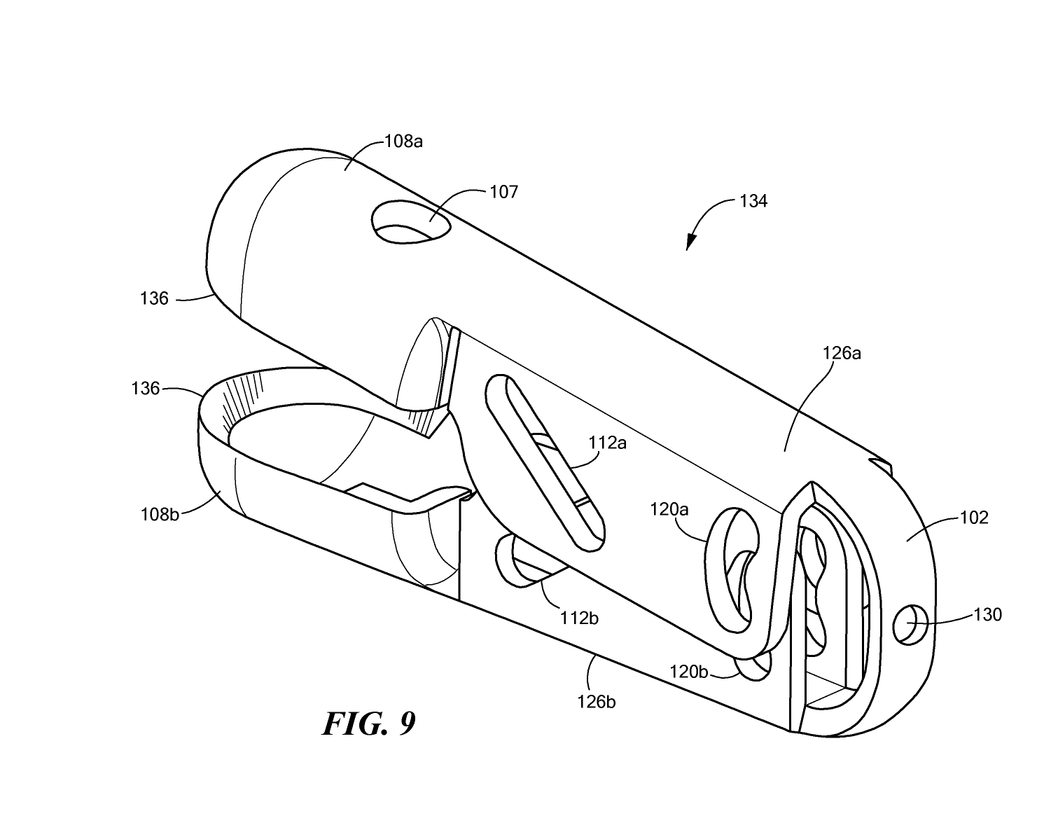

The jaws 108a, 108b may have peripheral edges 110a, 110b in the form of teeth that mesh when the forceps 134 are closed as shown in FIGS. 3-6 to provide for the cutting of tissue. Alternatively, referring the FIG. 9, the jaws of the biopsy forceps 134 may be provided with sharpened blade like edges 136 that abut one anther when the jaws are closed.

The self-hinge section 102 is bent around the centerline 116 to provide a hinge that is integral with the channel sections 126a, 126b and the jaws 108a, 108b. Following bending of the self-hinge section 102, the biopsy forceps 134 assumes the shape generally illustrated in FIGS. 3-8. The self-hinge section 102 includes an opening 130 that is sized to receive an actuation rod 132 as subsequently discussed.

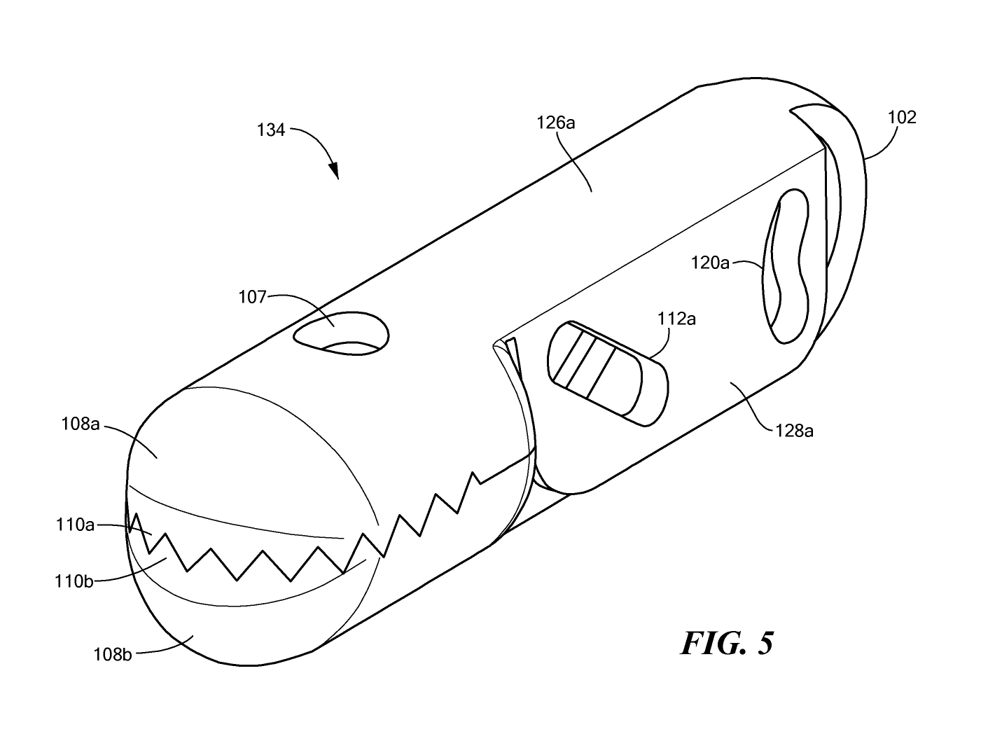

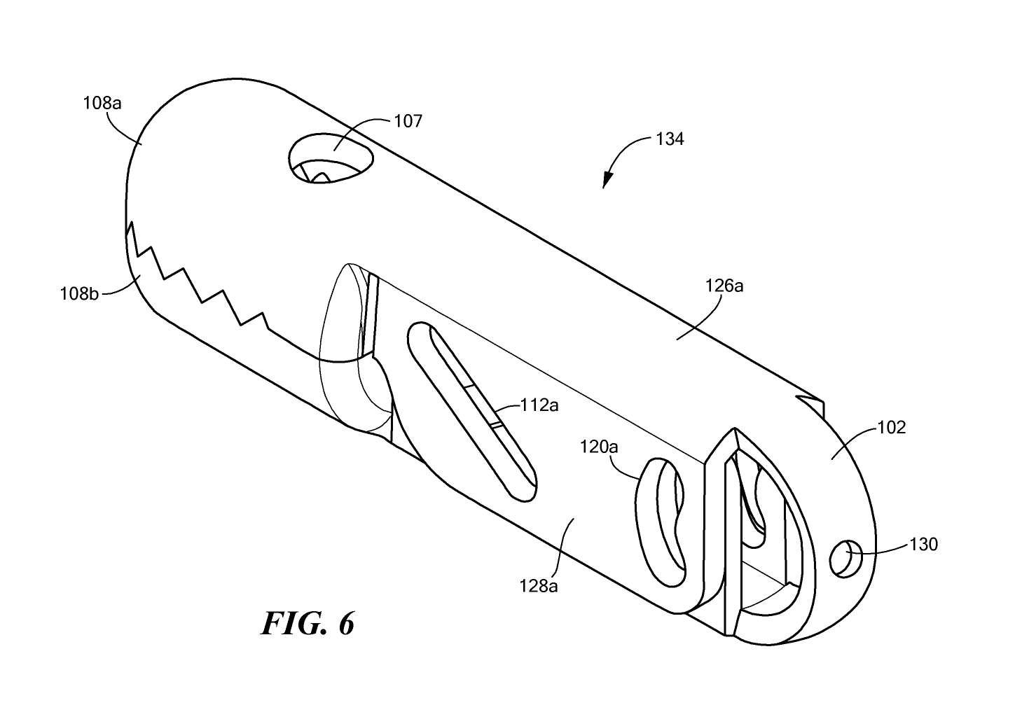

The biopsy forceps 134 are illustrated in FIGS. 3, 4 and 8 with the opposed jaws 108a, 108b opened and in FIGS. 5, 6 and 7 with the opposed jaws 108a, 108b closed. The biopsy forceps 134 thus have opposed jaw portions, each of which includes a jaw 108a, 108b and a channel section 126a, 126b respectively. The jaw portions are connected by the self-hinge 102 which is formed as a single unitary piece with the jaw portions.

Following the metal forming operations, each pair of slots 112a, 112b is disposed in opposed aligned within the side portions 128a, 128b of the channel sections 126a, 126b with the side portions 128a overlapping side portions 128b. Additionally, each pair of slots 120a, 120b is disposed in opposed aligned within the side portions 128a, 128b of the channel sections 126a, 126b. A close fit between the outer surfaces of the side portions 128b of the channel section 126b and the inner surfaces of the side portions 128a of the channel section 126a provides rigidity for the biopsy forceps 134 following the formation thereof.

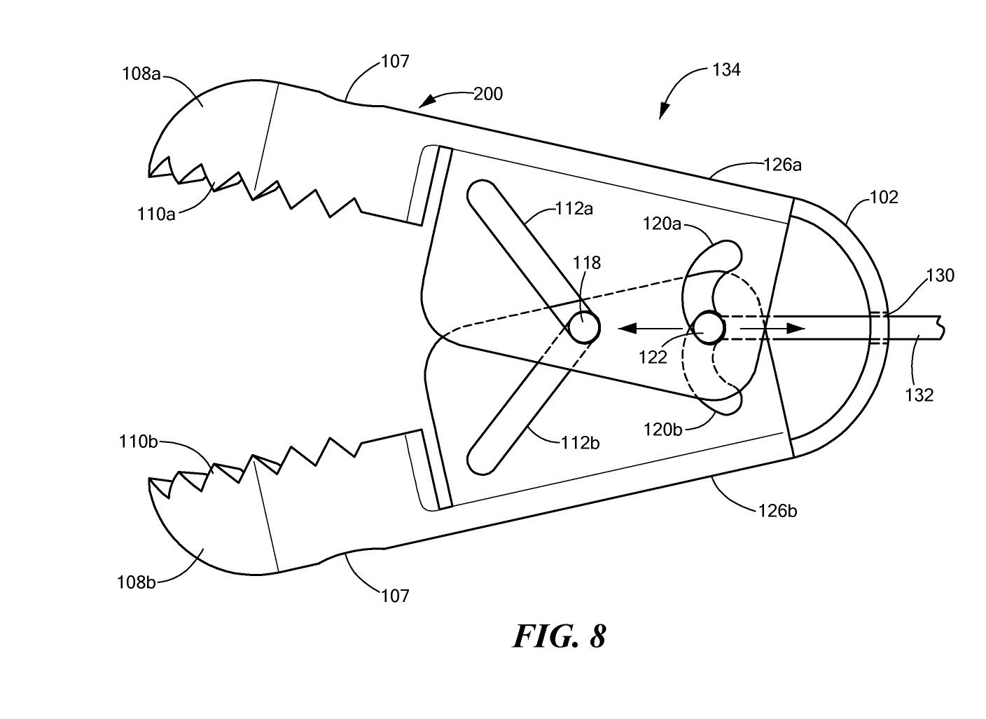

The fixed pin 118 extends through opposed pairs of slots 112a, 112b in the side portions 128a, 128b of the U-shaped channel sections 126a, 126b and is affixed at opposed ends to a surrounding tube or other supporting structure to prevent relative movement of the fixed pin 118 relative to the supporting structure while permitting slidable movement of the fixed pin 118 within, and relative to, the first slots 112a, 112b in the side portions 128a, 128b of the channel sections 126a, 126b. The movable pin 122 extends through both pairs of curvilinear slots 120a, 120b in the side portions 128a, 128b of the channel sections 126a, 126b more proximate the hinge section 102 than the first pairs of slots 112a, 112b as illustrated in FIGS. 7 and 8. The actuation rod 132 passes through the opening 130 in the hinge section 102 and is mechanically coupled to the movable pin 122. The movable pin 122 is thus movable longitudinally with respect to the fixed pin 118 by pushing or pulling the actuation rod 132. More specifically, to close the opposed U-shaped jaws 108a, 108b, the actuation rod 132 is pulled to increase the distance between the movable pin 122 and the fixed pin 118. Pulling of the actuation rod 132 thus causes the fixed pin 118 to slide within the first pairs of slots 112a, 112b, which, by reason of their respective angled orientations with respect to the longitudinal centerline 112, causes the opposed jaws 108a, 108b to close. Conversely, pushing of the actuation rod 132 toward the fixed pin 118 causes the movable pin 122 to move closer to the fixed pin 118 which, in turn, causes the fixed pin 118 to assume the orientation within the first pairs of slots 112a, 112b depicted in FIG. 8 to open the jaws 108a, 108b.

It should be appreciated that, in another embodiment, the pin 122 may be fixedly mounted as the fixed pin and the pin 118 may be provided as a movable pin. By coupling the actuation rod 132 to the pin 118 linear motion of the pin 118 with respect to the pin 122 may be achieved. In this alternative embodiment, pushing the actuation rod 132 toward the hinge section 102 causes the jaws 108a, 108b to close and pulling the actuation rod 132 away from the hinge section 102 causes the jaws 108a, 108b to open.

Furthermore, while actuation of the biopsy forceps is achieved in the illustrated embodiment via the use of the actuation rod 132 that passes through the opening 130 in the hinge section, opening and closure of the jaws may be achieved using one or more wires or rods that extends around the hinge section 102 rather than passing through an opening in the hinge section 102.

It will be appreciated by those of ordinary skill in the art that modifications to and variations of the above-described single piece forceps may be may without departing from the inventive concepts disclosed herein.

What is claimed is:

1. Biopsy forceps comprising:
a pair of opposed jaw portions, each jaw portion including a jaw and a generally U-shaped channel section having opposed side portions, the opposed jaw portions joined by a self-hinge, wherein the opposed jaw portions and the self-hinge are formed as a single unitary structure, the channel sections disposed between the jaws and the self-hinge;
each pair of opposed side portions including first and second pairs of opposed aligned slots sized to receive first and second pins respectively.

2. The biopsy forceps of claim 1 further including first and second pins, the first and second pins extending through the first and second pairs of opposed aligned slots respectively, the first and second pairs of opposed aligned slots being configured to produce closure and opening of the opposed jaws in response to relative linear movement of the first pin with respect to the second pin in first and second opposing directions respectively.

3. The biopsy forceps of claim 2 wherein one of the first and second pins is a fixed pin with ends thereof mounted to an adjacent supporting structure and the other one of said first and second pins is a movable pin that is movable with respect to the fixed pin; the biopsy forceps further including an actuator rod coupled to the movable pin, the movable pin being movable relative to the fixed pin in response to longitudinal movement of the actuator rod to cause the first pin to slidably move within the first set of slots so as to cause the jaws to open and close in response to the longitudinal movement of the actuator rod in first and second opposing directions respectively.

4. The biopsy forceps of claim 3 wherein the first sets of slots comprise elongated slots, the second sets of slots comprise curvilinear slots and the second sets of slots are located more proximate to the self-hinge than the second sets of slots.

5. The biopsy forceps of claim 4 wherein the elongated slots have first and second ends and are angled with respect to a forceps centerline located between the opposed jaw portions such that the first end each of the elongated slots is more proximate to the self-hinge than the second end of the respective elongated slot, and the second end of each of the elongated slots in opposed jaw portions is more distal from the forceps centerline than first end of the respective slot when the opposed jaws are opened.

6. The biopsy forceps of claim 5 wherein the first pin is the fixed pin and the second pin is the movable pin and the actuator rod is mechanically coupled to the second pin.

7. The biopsy forceps of claim 5 wherein the first pin is the movable pin and the second pin is the fixed pin and the actuator rod is mechanically coupled to the first pin.

8. The biopsy forceps of claim 1 wherein the opposed jaws have teeth that are configured such that the teeth mesh when the opposed jaws are closed.

9. The biopsy forceps of claim 8 wherein the teeth of the opposed jaws are serrated.

10. The biopsy forceps of claim 1 wherein at least one of the opposed jaws has a sharpened peripheral edge to provide for the cutting of tissue upon closure of the jaws.

11. A method of making biopsy forceps comprising the steps of:
forming a metal blank having a longitudinal centerline, the metal blank having a hinge portion with opposing ends, the longitudinal centerline extending through the hinge portion, the hinge portion having a hinge centerline orthogonal to the longitudinal centerline, the metal blank having first and second sections extending from opposing ends of the hinge portion, the first and second sections including first and second channel sections coupled to respective ends of the hinge portion and first and second jaw sections extending from the first and second channel sections respectively, the first and second channel sections each being generally symmetrical about the longitudinal centerline, the first and second channel sections each having a first pair of slots disposed generally symmetrically about the longitudinal centerline, the first and second sections including a second pair of slots disposed generally symmetrically about the longitudinal centerline, the first pair of slots being more distal from the hinge centerline than the second pair of slots;
forming first and second jaws respectively from the first and second jaw sections of the metal blank;
bending the first channel section along a pair of form lines substantially parallel to and generally symmetrically spaced on from the longitudinal centerline by a first distance to form a first generally U-shaped channel section having opposed first side portions;
bending the second channel section along a pair of form lines substantially parallel to and generally symmetrically spaced on from the longitudinal centerline by a second distance that is less than the first distance to form second generally U-shaped channel section having opposed second side portions; and
bending the hinge portion about the second centerline so that the first and second jaws are in opposed aligned relation with the side portions of the second generally U-shaped channel section disposed at least partially between the side portions of the first generally U-shaped channel section.

12. The method of claim 11 wherein the first pair of slots comprise a first pair of elongated slots and the second pair of slots comprise a second pair of curvilinear slots.

13. The method of claim 11 further including, following the bending steps:
inserting a first pin having opposing ends through the first pairs of slots and inserting a second pin having opposing ends through the second pairs of slots; and
fixing the opposing ends of one of the first and second pins to an adjacent supporting structure and mechanically coupling an actuator rod to the other one of the first and second pins.

14. The method of claim 13 wherein the fixing step comprises the step of fixing the opposing ends of the first pin to an adjacent supporting structure and mechanically coupling the actuator rod to the second pin.

15. The method of claim 14 wherein the metal blank forming step includes the step of forming an opening in the hinge portion sized to slidably receive the actuator rod, and, the mechanically coupling step includes the step of slidably disposing the actuator rod through the opening in the hinge portion.

16. The method of claim 13 wherein the fixing step comprises the step of fixing the opposing ends of the second pin to an adjacent supporting structure and mechanically coupling the actuator rod to the first pin.

17. The method of claim 14 wherein the metal blank forming step includes the step of forming an opening in the hinge portion sized to slidably receive the actuator rod, and, the mechanically coupling step includes the step of slidably disposing the actuator rod through the opening in the hinge portion.

18. The method of claim 11 wherein the jaw sections have peripheral edges, the method further including the step of sharpening the peripheral edges of the jaw sections.

19. The method of claim 12 wherein the jaw sections have peripheral edges, the method further including the step of forming teeth on the peripheral edges of the jaw sections.

20. The method of claim 19 wherein the step of forming teeth on the peripheral edges of the jaw sections includes the step of forming teeth oriented and configured to mesh upon closure of the first and second opposed jaws of the biopsy forceps.
